(19) United States
(12) Patent Application Publication
(10) Pub. No.: US 2026/0355476 A1
(43) Pub. Date: Nov. 12, 2026

(54) BALLOON CATHETER FOR SELECTIVE SIDE VESSEL INFUSION

(71) Applicant: INCITE MEDICAL SARL, Geneva (CH)

(72) Inventors: Alexandre Ioan Romoscanu, Geneva (CH); Daniel Cosovanu, Marginea (RO)

(21) Appl. No.: 19/147,579

(22) PCT Filed: Jan. 12, 2024
(86) PCT No.: PCT/IB2024/050344

§ 371 Date: Jul. 11, 2025

Related U.S. Application Data
(60) Provisional application No. 63/439,007, filed on Jan. 13, 2023.

(51) Int. Cl. A61M 25/10

(52) U.S. Cl. CPC A61M 25/1011; A61M 2025/1013; A61M 2025/1045; A61M 2025/105; A61M 2025/1052; A61M 2025/1079; A61M 2025/1086; A61M 2025/1095; A61M 2202/0007; A61M 2202/04; A61M 2205/3306; A61M 2210/12

(57) ABSTRACT

A catheter 100 assembly that includes two overlapping, coaxial, distally located balloons 201, 203 that facilitate access for fluid injection to a lateral branch of a given vessel, and may also occlude the flow inside said vessel, thus allowing directed injection of another fluid inside said lateral branch. The inner balloon 201 is sealed and may be inflated by injection of fluid via a lumen inside the catheter. The outer balloon 203 acts as a sleeve for the inner balloon and mimics the volume movements (inflation and deflation) of the contained one. Moreover, the outer balloon may include one or several radial ports 209 that can be used to direct another fluid that is to be injected inside the targeted lateral branch.

### PRIORITY CLAIM

This patent application is a National Phase entry of PCT Application No. PCT/IB2024/050344, filed Jan. 12, 2024, which claims the benefit of U.S. Provisional Application No. 63/439,007 filed Jan. 13, 2023, the disclosures of which are incorporated by reference herein in their entireties.

### FIELD OF THE DISCLOSURE

The present disclosure relates generally to a catheter, and more specifically to a balloon catheter for chemical ablation.

### BACKGROUND

Catheters are medical devices that can be inserted inside the body, through the vasculature. They are used to perform various surgical procedures and to treat different diseases. A balloon catheter is a type of catheter that contains one or several compliant and inflatable parts, usually located at the distal end of the device. The catheter is initially introduced in the body with the balloon in a deflated state. Upon insertion and positioning in the target vasculature location, all or some of the catheter balloons are inflated in order to perform certain procedures. Positioning other percutaneous devices, such as guidewires or catheters in a particular location inside the body accessible through the vasculature, is also possible.

### SUMMARY

Various embodiments of the present disclosure relate to a catheter assembly that includes two overlapping, coaxial, distally-located balloons that facilitate access for fluid injection to a lateral branch of a given vessel, and may also occlude the flow inside said vessel, thus allowing directed injection of another fluid inside said lateral branch. The two balloons are coaxially placed and have the same dimensions or are similar in size. The inner balloon is sealed and may be inflated by injection of fluid (saline, contrast fluid, or others, potentially air or a gas) via a lumen inside the catheter. The outer balloon acts as a sleeve for the inner balloon and mimics the volume movements (inflation and deflation) of the contained balloon. Moreover, the outer balloon may include one or several radial ports that can be used to direct another fluid that is to be injected inside the targeted lateral branch. Said fluid is fed inside the interstitial space delimited by the inner balloon outer diameter (OD) and the outer balloon inner diameter (ID) by way of a dedicated lumen included in the catheter shaft and can escape said space through the above-mentioned radial port or ports.

The catheter assembly is considered for use in cardiac procedures, such as those used for the treatment of atrial fibrillation, ventricular tachycardias and more generally arrythmias, but not limited to these procedures.

Embodiments of the disclosure include a catheter having a shaft, an inflation balloon, and an infusion balloon. The shaft includes an inflation lumen and an infusion lumen. The inflation balloon has a proximal end and a distal end, each of the ends secured to an outer surface of the shaft. The infusion balloon has a proximal end and a distal end, each of the ends sealed to the outer surface of the shaft. The proximal end of the infusion balloon is secured proximally of the inflation balloon and the distal end of the infusion balloon is secured distally of the inflation balloon. The shaft includes an inflation port in fluid communication with the inflation lumen and an infusion port between the distal end of the inflation balloon and the distal end of the infusion balloon. The inflation balloon may be configured to occlude a vessel when inflated.

According to embodiments of the invention, a catheter includes a shaft with an inflation lumen and an infusion lumen. A dual layer balloon is mounted to an exterior surface of the catheter shaft. The dual layer balloon includes an infusion port. An imaging device is located within an interior chamber of the dual layer balloon. The imaging device is positioned to capture an image through the infusion port. The imaging device may be attached to the catheter shaft. A light source may be attached adjacent to the imaging device.

Embodiments of the invention also include a catheter having a catheter shaft, an anchor balloon, a coaxial double balloon, and a camera. The catheter shaft has an inflation lumen and an infusion lumen, and a guidewire extends through the catheter shaft. The coaxial double balloon has an infusion port in fluid communication with the infusion lumen. The coaxial double balloon is configured to translate axially in relation to the anchor balloon. The camera is located within an interior chamber of the balloon, the camera positioned to capture an image through the infusion port.

### BRIEF DESCRIPTION OF THE DRAWINGS

The present invention will become more fully understood from the detailed description given herein below for illustration only, and thus not limitative of the present invention, wherein:

FIG. 1 is a perspective view of a primary vessel, a side vessel, and a catheter positioned within the primary vessel, according to an embodiment of the disclosure;

FIG. 2 is a perspective partial view of a catheter, according to an embodiment of the disclosure;

FIG. 3A is a perspective partial view of a catheter with a balloon having a circular port, according to an embodiment of the disclosure;

FIG. 3B is a perspective partial view of a catheter with a balloon having a series of oval ports, according to an embodiment of the disclosure;

FIG. 3C is a perspective partial view of a catheter with a balloon having an oval port, according to an embodiment of the disclosure;

FIG. 3D is a perspective partial view of a catheter with a balloon having a plurality of circular ports, according to an embodiment of the disclosure;

FIG. 4 is a perspective partial view of a catheter with a balloon having a marker band around a port, according to an embodiment of the disclosure;

FIG. 5A is a perspective partial view of a catheter with an outwardly extending balloon port, according to an embodiment of the disclosure;

FIG. 5B is an enlarged view of the balloon port of FIG. 5A;

FIG. 6 is a perspective partial view of a catheter with a balloon port connected to a lumen, according to an embodiment of the disclosure;

FIG. 7 is an enlarged partial view of the catheter of claim 6 depicting the lumen in an interstitial space between an outer balloon and an inner balloon, according to an embodiment of the disclosure;

FIG. 8 is a perspective partial view of a catheter with an imaging device and LED light positioned within a balloon, according to an embodiment of the disclosure;

FIG. 9 is a perspective sectional view of a catheter with an imaging device depicting the flow of inflation and infusion fluids, according to an embodiment of the disclosure;

FIG. 10 is a side sectional view of a catheter and a proximal fitting, according to an embodiment of the disclosure;

FIG. 11A is a fragmentary perspective view of a catheter equipped with an imaging device, according to an embodiment of the disclosure;

FIG. 11B is a side sectional view of a catheter equipped with an imaging device inside the vasculature, according to an embodiment of the disclosure;

FIG. 12A is a side sectional view of a catheter equipped with an imaging device inside the vasculature, according to an embodiment of the disclosure;

FIG. 12B is the catheter of FIG. 12A with a balloon in an inflation and with motion to the right;

FIG. 13 is a multi-sheath balloon catheter with a coaxial double balloon on an inner sheath and a secondary balloon on an outer sheath, according to an embodiment of the disclosure;

FIG. 14A is a multiple balloon catheter with a distal anchor balloon, a centrally located coaxial double balloon on an inner sheath, and a proximally positioned ramification blocking balloon on an outer sheath, according to an embodiment of the disclosure;

FIG. 14B is a multiple balloon catheter positioned within the vasculature, according to an embodiment of the disclosure;

FIG. 15A is a perspective view of a balloon having a blood transfer passage, according to an embodiment of the disclosure;

FIG. 15B is an end view of the balloon of FIG. 15A;

FIG. 15C is a perspective view of a balloon having an enlarged blood transfer passage, according to an embodiment of the disclosure;

FIG. 15D is an alternative perspective view of the balloon of FIG. 15C;

FIG. 16A is a perspective view of a balloon having a blood transfer lumen, according to an embodiment of the disclosure;

FIG. 16B is an end view of the balloon of FIG. 16A;

FIG. 17A is a perspective view of a catheter having a blood transfer lumen, according to an embodiment of the disclosure; and

FIG. 17B is a perspective view of the catheter of FIG. 17A depicting the blood transfer lumen passing through an interior of a balloon.

While the embodiments of the disclosure are amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

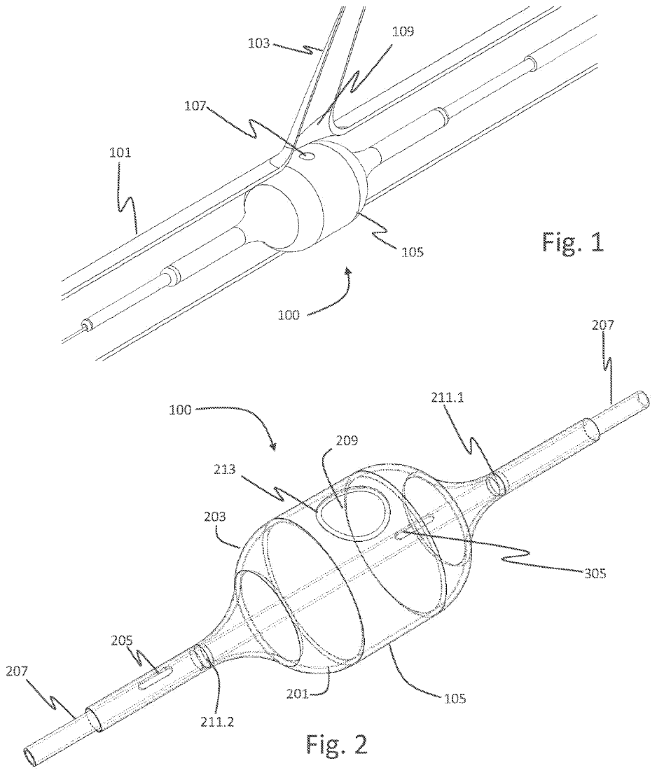

Various embodiments of the disclosed catheter assembly 100 can be used in a main or primary vasculature 101 that has a lateral branch 103, however, for clarity/simplicity only the case of the coronary sinus (CS) as the main vessel 101 and oblique vein of Marshall (OVM) as the lateral branch 103 is illustrated, as presented in FIGS. 1A and 1B. The catheter assembly 100 may be generally positioned within the CS 101 such that a balloon 105 occludes or partially occludes the CS 101 while a port 107 in the balloon 105 is located at the ostium 109 of the OVM 103.

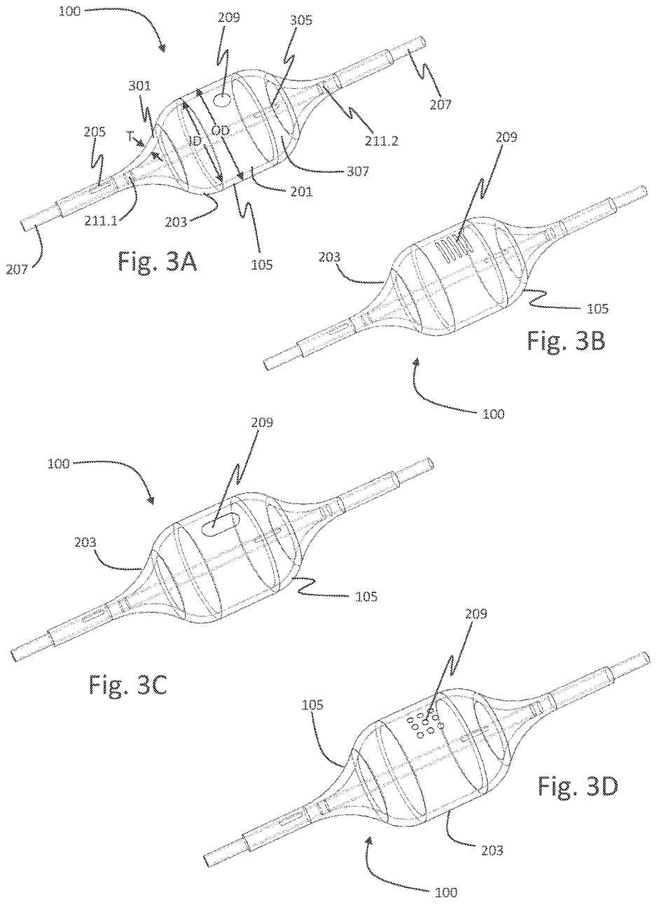

Referring to FIG. 2 and FIGS. 3A-3D, an overlapping double balloon 105 is depicted which may include an inner balloon 201 and an outer balloon 203. For purposes of illustration, the outer balloon 203 in FIG. 2 is depicted as translucent to better show the inner balloon (IB) 203 and catheter shaft 207. The inner balloon 201 is fully sealed and may be inflated through a lumen of the catheter shaft 207. The inner balloon 201 may attach to an outer surface of the catheter shaft 207. The ends of the inner balloon 201 may include seals 211.1, 211.2. Such seals 211 may secure the ends of inner balloon 201 to the catheter shaft 207 to prevent axial leakage. In some embodiments, seal 211 may be, or contain, a marker band such as a radiopaque band or a magnetic band. An inflation port 305 in the catheter shaft 207 is positioned within an interior chamber 307 of the inner balloon 201 and may be used to inflate or deflate the balloon.

An outer balloon (OB) 203, overlapping the first balloon 201, has one or several lateral exit ports 209 for radial fluid injection (such as ethanol). Port openings may be between 0 and 8 mm at their widest points. The space 301 delimited by the inner balloon 201 outer diameter (OD) and the outer balloon 203 inner diameter (ID) is connected to a lumen of the catheter shaft 207 via infusion port 205 through which the infused fluid, such as ethanol, may flow towards the radial exit port or ports 209. The space 301 delimited by the inner balloon 201 outer surface and the outer balloon 203 inner surface may have a deminimis thickness T when no fluid is injected, flowing therethrough, or is stationary therein. Thickness refers to a maximum distance between the outer surface of the inner balloon and the outer balloon. Such a gap, or space 301, may vary at various stages of a procedure. In some embodiments, the outer balloon may be omitted. The catheter main body 207 may include one or several extrusions with several lumens and lateral ports used for the interaction with the balloons. In some embodiments, the outer balloon 203 may have a reinforcing structure or thickened edge 213 about the port 209. In some embodiments, the reinforcing structure 213 may be a marker band.

FIGS. 3A-3D depict embodiments of overlapping double balloons having an interstitial free fluid path and ports with no radial port marker. FIG. 3A depicts an embodiment of a catheter 100 with an overlapping double balloon 105 having a single external circular port 209 in outer balloon 203. FIG. 3B depicts an embodiment of an overlapping double balloon 105 with multiple external slotted ports 209 in outer balloon 203. FIG. 3C depicts an embodiment of an overlapping double balloon 105 with a single external slot port 209 in outer balloon 203. FIG. 3D depicts an embodiment of an overlapping double balloon 105 with multiple external hole ports 209 in outer balloon 203.

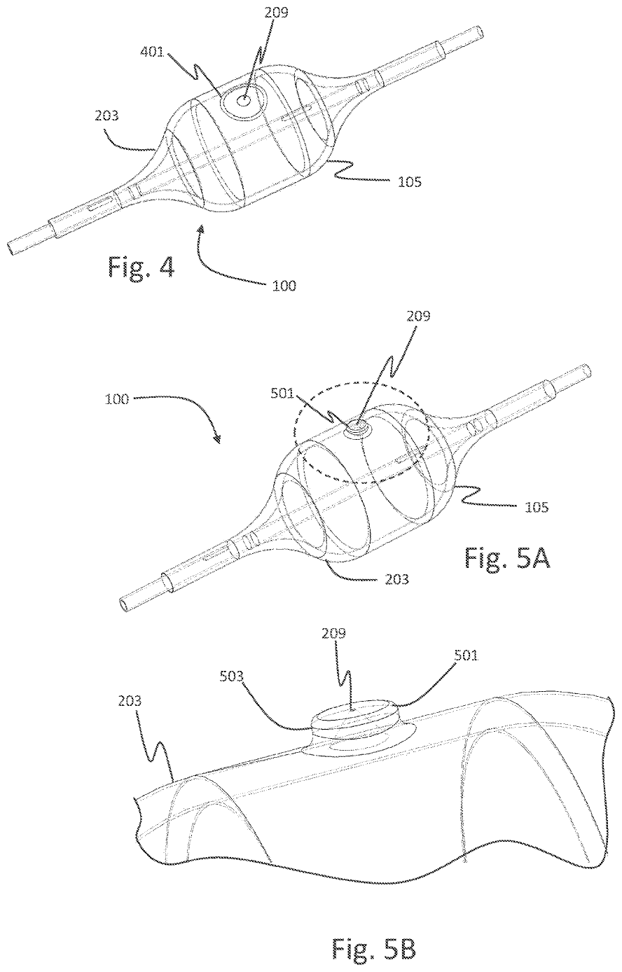

FIGS. 4, 5A, and 5B depict embodiments of overlapping double balloons having an interstitial free fluid path and ports with a radial port marker. Referring to FIG. 4, outer balloon 203 includes a single circular port 209 with a thickened balloon layer radial port marker 401 placed at a distance from the port 209. Referring to FIGS. 5A and 5B, overlapping double balloon 105 has an outer balloon 203 with a single needle hole external hole port 209 encompassed in a lateral compliant extrusion 501 that extends outside as a sleeve before reaching the maximum pressure needed for injecting the fluid inside the vasculature. The extrusion may also include a radial port marker 503.

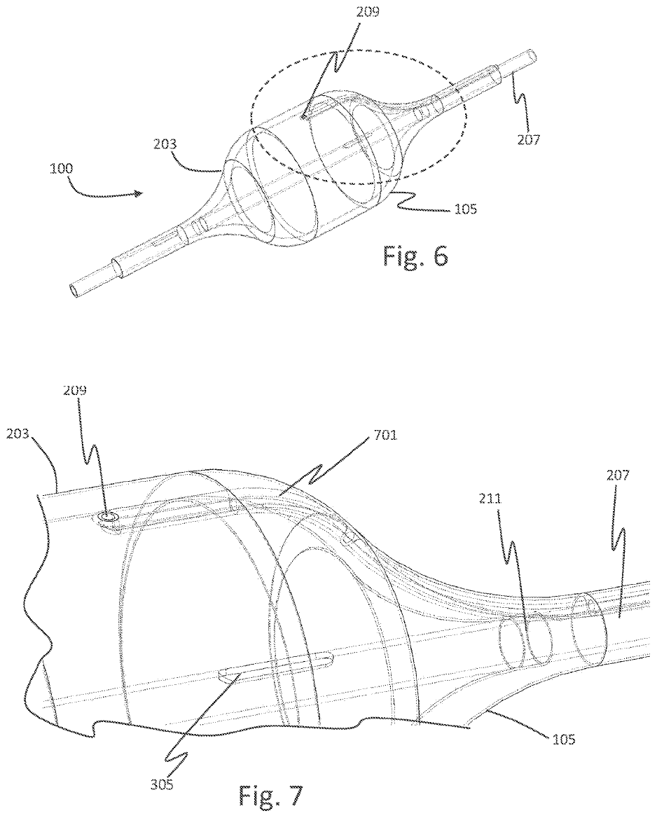

Referring to FIGS. 6 and 7, an embodiment of a distal balloon assembly 105 is shown with an outer balloon 203 having a single circular radial port 209 and a tubular fluid path. The infusion fluid goes along the lumen 701 attached to the inner wall of the outer balloon 203 that connects the exit port 209 of the outer balloon 203 and the injection port of the catheter. As a note, the guide tube 701 for the infusion fluid, in another embodiment, may be free, being only attached to the inlet and the outlet. An alternative embodiment is similar to the above, but the lumen is located on the outer edge of the inner balloon, and no outer balloon is present.

As the inner balloon needs to deform less and the outer balloon must take the shape of the first one, the balloons may preferably be manufactured from different materials as to show the following properties: the inner balloon is non-compliant or semi-compliant and the outer balloon is semi-compliant or compliant. Both balloons may be characterized by several properties such as the following. Both balloons may have substantially similar size. For example, both balloons may have a similar layer thickness and a maximum outer diameter when inflated. Both balloons may have similar shapes. For example, the balloons may be spherical, elongated, conical, offset, tapered, or other such shapes as known in the art and suitable for medical procedures in the vasculature. A gap layer between the balloon walls (in the deflated state) may be characterized as having no gap (with the outer balloon stretched on the inner balloon), or with a small gap. A minimum pressure may be needed to access the exit port of the outer with the injection fluid when the inner balloon is inflated.

The outer balloon may be characterized in that the external port(s) may be one or several ports. The outer balloon may include 1, 5, or 9 external ports. The outer balloon may include between 1 and 12 ports. A shape of the one or several external port(s) may be, for example, circular, oval, slot, needle holes, etc. Other shapes, or combinations of shapes, are not beyond the scope of this disclosure. In embodiments, the outer balloon may include the presence of a radial port marker with or without a thickened or reinforced portion of the balloon layer in the proximity of the external port(s) that prevents tearing. Radial port markers may include, for example a ring, ridge or lip circumventing the radial aperture. The radial port marker may be positioned, for example, on the edge of the port(s) or at a small distance up to the edge of the port(s). The radial port marker may be, for example, a ring, slot of the balloon layer, attached outer ring, an embedded thread, potentially containing radiopaque materials such as but not limited to BaSO4, PtIr, or tungsten. Embodiments of the radial port marker may include a thickened balloon layer, attached stiffening material (ring etc.), embedded thread, etc.

As disclosed above and discussed in more detail below, a fluid path for an infusion fluid to exit the ostium at the external port may be either an interstitial path through space 301 or a tubular path through lumen 701. Regarding the interstitial, free path, the fluid flows between the outer surface of the inner balloon and the inner surface of the outer balloon, i.e. interstitially, until exiting a radial exit port of the outer balloon. This embodiment is illustrated, for example in FIGS. 1 to 6, and FIGS. 8-10 for conciseness reasons, and without excluding alternatives, such as the tubular path below. In some embodiments, the outer diameter of the inner balloon, and/or the inner diameter of the outer balloon includes small ridges, bumps, or other features, which prevent the interstitial space between the outer diameter of the inner balloon, or the inner diameter of the outer balloon to have a zero thickness even in absence of infused fluid pressure. These features may improve the interstitial flow between the outer diameter of the inner balloon, and the inner diameter of the outer balloon, as well as in the vicinity of the radial exit port(s).

Regarding the tubular path, infusion fluid flows is inside a lumen 701 that connects the lateral injection port 205 of the catheter 207, or the fluid inlet 1003 at the handle or proximal fittings 1001, and the exit port 209 of the outer balloon 203. In this case, the outer balloon 203 itself is optional, with the catheter assembly includes a single balloon, fitted with said lumen 701 embedded in, or inside, or outside of the balloon membrane, or free within or outside of the inner balloon. A possible embodiment is illustrated in FIGS. 7A and 7B, which is described in detail above. Said lumen 701 may be created in different ways. For instance, an easily demoldable (PTFE coated, for instance) mandrel may be applied on the outer diameter of the inner balloon 201, and a layer of outer balloon-adhering polymer may be included to cover the mandrel. Upon polymerization, curing or drying of said polymer, the mandrel is removed, resulting in said lumen 701.

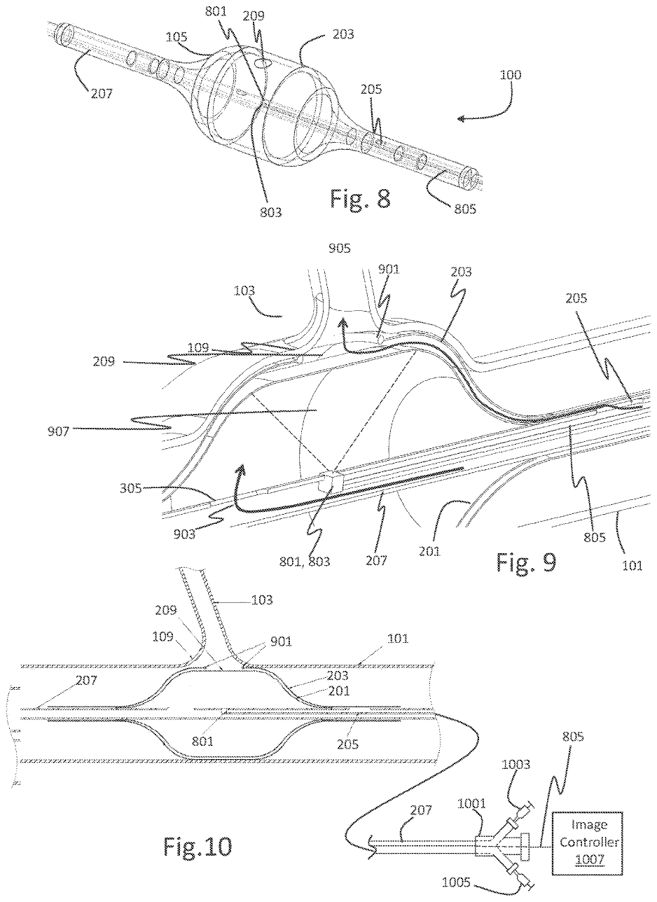

Various embodiments of the catheter assembly may include other aspects. FIGS. 8-12 depict embodiments of a catheter having an imaging device. In some embodiments, the catheter may further include a light source. Referring to FIG. 8, a camera 801 (CCD or CMOS, such as, but not limited to Omnivision™ OCH or OVM series, with appropriate aperture and lens) may be attached to the main body of the catheter 207, placed inside the two balloons 105 and, next to the camera (or the same block body), a light source 803 (such as, but not limited to, a LED) may be included. The inner balloon 201 and optionally the outer balloon 203 may be translucent to facilitate capturing images. The imaging device 801 may be oriented radially, or axially, with a mirror for radial imaging). Moreover, the imaging device 801 and light source 803 system are connected to the corresponding wiring 805 (electrical and/or optic). As depicted in FIG. 9, this system 100 allows the visualization 907 through the inflated balloon 105 of the ostium 109 of the lateral branch 103 in a direct manner. This simplifies and facilitates the positioning and orientation of the lateral port(s) 209 of the outer balloon 203 and also allows the user to reduce the needed contrast fluid injection. Indeed, the appropriate catheter positioning may be obtained by overlapping the radial port of the outer balloon 203, optionally surrounded by a delimiter (ridge, or camera visible edge) 901, and the visible ostium 109, under the assumption that the ostium 109 is identified by the blood, which contrasts with the inner wall of the main vessel 101, which is in close contact the outer diameter of the outer balloon 203, and whose aspect may differ from the ostium blood under adequate lighting from the light source 803.

FIG. 9 further depicts flows of infusion fluid 903 and inflation fluid 905 from the catheter 207. Inflation fluid may travel through a first lumen of the catheter 207 and exit through inflation port 305, thereby inflating the inner balloon 201. In embodiments, inflation fluid may be a clear fluid, such as a solution, to prevent interference with the visualization 907 from the imaging device 801. Infusion fluid 903 may exit the catheter 207 through the infusion port 205 and travel through the interstitial space between the inner balloon 201 and the outer balloon 203, and exit into the ostium 109. Examples of infusion fluid include, for example, alcohol, contrast dye, or other such fluids commonly delivered to the vasculature during medical procedures. Referring to FIG. 10, the catheter may be in fluid communication with an infusion fluid source 1003 and an inflation fluid source 1005. In some embodiments, the catheter 207 may connect with proximal fittings 1001 to facilitate connections with fluid sources, other tools, or the delivery of additional devices during a procedure. Wiring 805 may exit a proximal end of the catheter 207 and connect to, for example, an image controller 1007. Image controller 1007 may further include or link to displays such as those used during medical procedures.

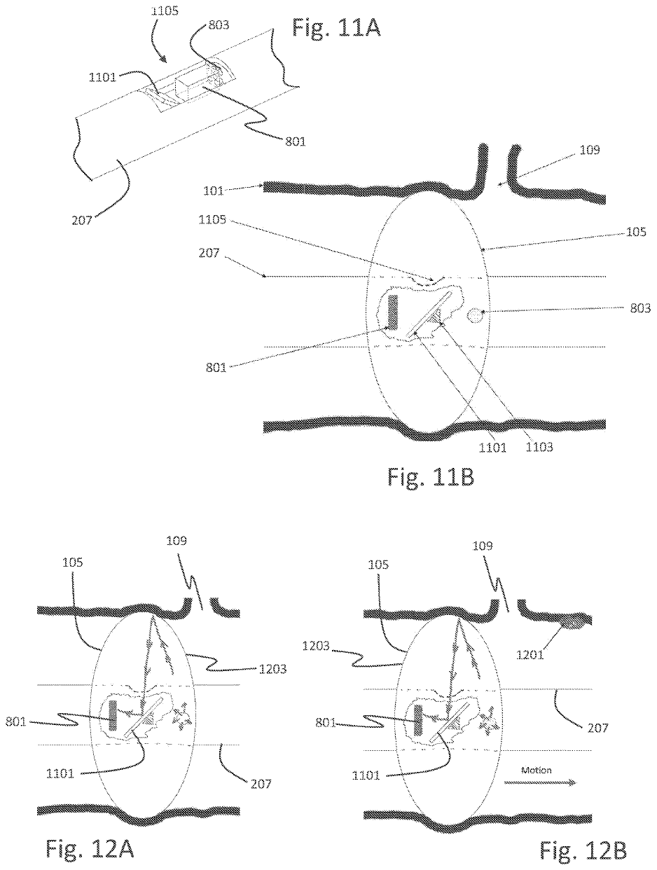

In various embodiments, an imaging device may be installed inside the coaxial double balloon to capture images for positioning information or for diagnosis. The imaging system may be equipped with a mirror and an orientation system, which could move the mirror (or the imaging device) in order to capture images from different angles through a sweeping motion; or the imaging device may remain in a fixed position and the sweeping motion created by rotating the catheter proximally from a handle. The imaging device installed in the balloon catheter, referring to FIGS. 11 and 12, may include the following: a catheter main body 207, transparent/translucid balloon(s) 105, an imaging device 801, an optional mirror 1101, an optional mirror orienting and positioning mechanism 1103, a light source 803, and an optional radial aperture 1105.

In the embodiment presented in FIG. 11, at the inflation of the balloon(s) 105 the membrane pushes the blood inside the vasculature, and as the balloon(s) 105 volume expands, the balloon(s) 105 may reach the vasculature walls 101. With the help of a small light source 803 (LED, fiber optic or other) the volume contained inside the vasculature 101 is illuminated together with the surfaces that the balloon(s) 105 membrane is in contact with. Then the imaging device 801 located inside the balloon(s) 105, attached to the catheter main body 207, may capture images either directly (without the mirror 1101) or through use of the mirror 1101, that may be equipped with an orienting and positioning mechanism 1103.

The radial aperture 1105 plays the role of a small window that lets the light reach inside the catheter main body 207, which is suitable for the imaging device 801 and mirror 1101 with a fixed configuration. The aperture 1105, may also be replaced by a transparent ring placed on the catheter main body 207 to permit the image recording up to a coaxial rotational motion or even more if the mirror orienting and positioning mechanism 1103 may also change inclination angle. Alternatively, the shaft may be transparent.

FIG. 12A depicts an example of image capturing of the vasculature wall 101 inside the balloon 105 of catheter 207. FIG. 12B shows a catheter 207 with camera 801 in motion to the right with balloon 105 in an inflation state, where an anatomical feature with a medical relevance 1201 is shown along the vessel wall. In FIG. 12B, the motion of a balloon 105 equipped with a camera 801 is illustrated. The mirror 1101 helps the camera capture images of the top of the balloon 105. As the balloon membrane 1203 touches different surfaces, the image changes accordingly. Thus, the FIG. 12B embodiment may be used as a positioning system in order to determine if the port 209 is placed next to or onto an ostium 109, that leads to a side branch from the main vasculature 101, or other anatomical reference with a medical relevance (lesion, valve etc.) 1201. The direct visual observation from the inside of the vascular tree may facilitate the positioning and orientation of the catheter with reduced, or in some cases, without any use of contrast fluid. The ostium of a side vessel, such as the OVM, may for instance appear as a spot whose color differs from the one color of the main vessel wall, which is in intimate contact with the device balloon OD.

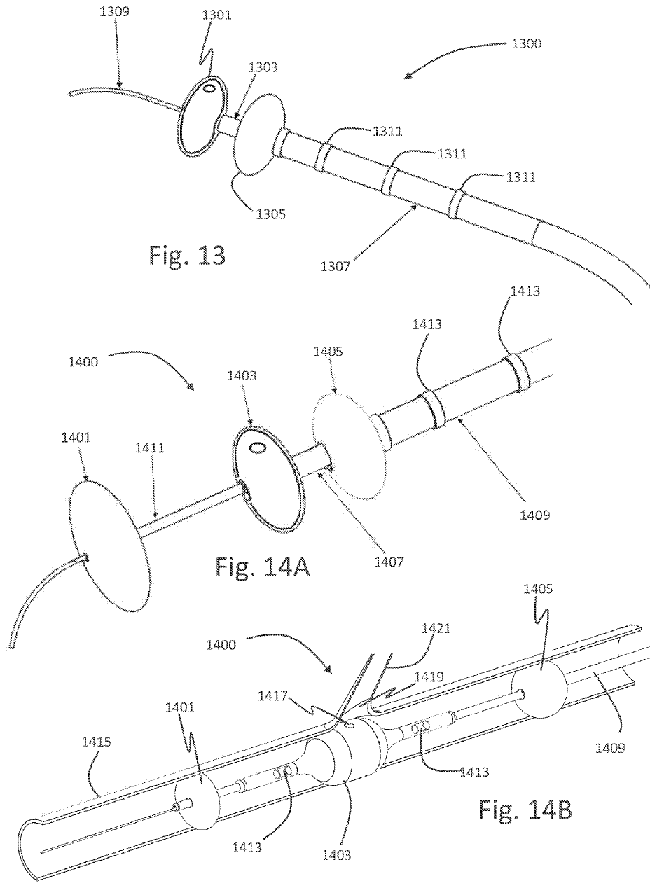

FIG. 13 shows a multi-sheath balloon catheter 1300 where the coaxially double balloon 1301 is placed on the inner sheath 1303 and the additional balloon 1305 is placed on the outer sheath 1307, being guided over the guide wire 1309. In the literature and the practical medical experiences, for the case of the OVM chemical ablation infusion (but not being limited to), there is a chance of finding ramifications that lead back to the CS. In these cases, the procedure of chemical ablation is not as successful (or not successful at all) due to the dilution of the alcohol inside the vasculature connected to the OVM. In order to prevent this from happening, an additional balloon may be included (which may optionally be mobile, relative to the infusion balloon) that can be placed on the ostium of the ramification in order to block access of the ramification back to the CS, allowing the procedure to be performed in an isolated manner.

In FIG. 13 an embodiment of such a catheter 1300 is presented that has a double sheath: the inner one 1303 is used for the positioning of the coaxial double balloon 1301 for the identification of the OVM ostium, and the outer sheath 1307 is used for the positioning of the additional mobile balloon 1305 next to the ostium of the ramification that leads back to the CS. For enhanced positioning, the outer sheath 1307 may include several marked bands 1311. The entire system is passed along the pre-positioned guidewire 1309. According to the purpose and the location of the medical procedure, various embodiments include variations such as single or multiple sheaths, balloon placement, and additional balloons.

In a single sheath embodiment, the coaxial double balloon 1301 and the additional balloon 1305 may be fixed on the same sheath, as the ostium of a ramification of the side vessel of the main vessel may be placed in the proximity of the ostium of the side vessel the additional balloon 1305 may be a long balloon that can cover a long area or multiple balloons could be placed on the catheter. For the multiple sheath embodiments, the additional balloon(s) 1305 may be mobile and the blocking of the ramification being facilitated. The additional balloon 1305 may be placed proximally to the coaxial double balloon 1301 or distally.

Optionally, the additional balloon 1305 or another balloon may act as an anchoring balloon. Said anchoring balloon (or anchor), when inflated, may be used for providing a stable fixture inside the blood vessels that can be located (through the use of the radiopaque markers/marker bands) and that can be considered as the reference for the relative position of the other balloons 1301, 1305 located proximally or distally to said anchoring balloon. In some embodiments, the anchor would be the most proximal or the most distal balloon. Considering that the sheaths axial compliance is negligeable in absence of large axial forces, the axial movement of the sheaths (on which the balloons are fixed) relative to the anchor balloon may be controlled with the desired precision through the mechanism of the handle. In some embodiments, the anchoring balloon can also inflate inwards (i.e., designed to reduce the inner diameter of the carrying sheath upon inflation) in such a way as to block the axial mobility of a guide wire going through said sheath. In this way, the anchoring balloon may fix the catheter assembly at a position on the guide wire that can be used as a reference for a positioning of the other mobile parts of the balloon catheter. Thus, the positioning system based on the anchor balloon provides accurate positioning of the balloons inside the main vein and provides a method for adjusting the location of the coaxial double balloon with access to a lateral vein, branched from a main vein, through which a medical procedure can be performed such as a chemical ablation.

FIG. 14A shows a multiple balloon catheter embodiment 1400 with a balloon anchor 1401 distally, the coaxial double balloon 1403 in the middle and the additional ramification blocking balloon 1405 proximally. The coaxial double balloon 1403 being placed in the middle on the second sheath 1407, and additional balloon for the ramifications blocking proximally 1405 on the outer sheath 1409, and a balloon anchor 1401 on the inner guide sheath 1411. For the case of the OVM, the distal balloon anchor 1401 may be fixed inside the CS distally to the OVM ostium and the next balloons 1403, 1405 may move axially relative to the distal balloon anchor 1401 in order to locate the OVM ostium next to the coaxial double balloon 1403. Marker bands 1413 may further assist with positioning the device. In some embodiments, the anchoring balloon can also be placed proximally to the coaxial double balloon. Referring to FIG. 14B, a multiple balloon catheter is depicted within the vasculature 1415 such that a port 1417 of the coaxial double 1403 is positioned at an ostium 1419 of a branch vessel 1421.

Various embodiments may also incorporate the following non-limiting aspects. A blood transfer lumen coaxial to the catheter shaft, as the inflated balloons occlude the vasculature, that allows the blood circulation distally and proximally to the balloons. In order to allow the blood circulation distally and proximally to the inflated balloons, an embodiment may feature a less compliant structure extending axially between the distal and proximal seals of the balloons, which would prevent said balloons to inflate in an axially symmetric manner. If this less compliant feature is positioned in the balloon side opposite to the side vessel, a channel allowing the blood circulation distally and proximally to the inflated balloons would result. For example, the balloons may include a thicker or hardened side which, when the balloons are inflated, creates a gap that allows the continuous circulation in the main vasculature.

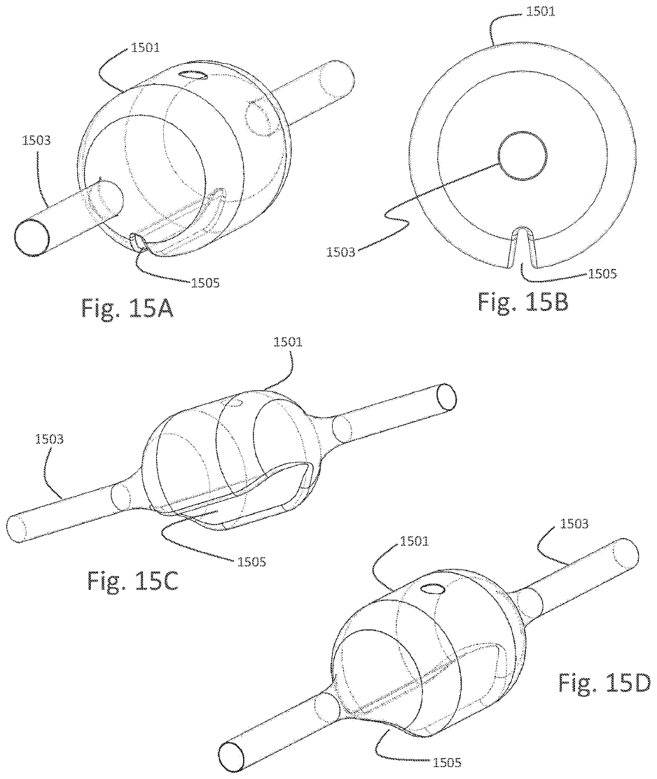

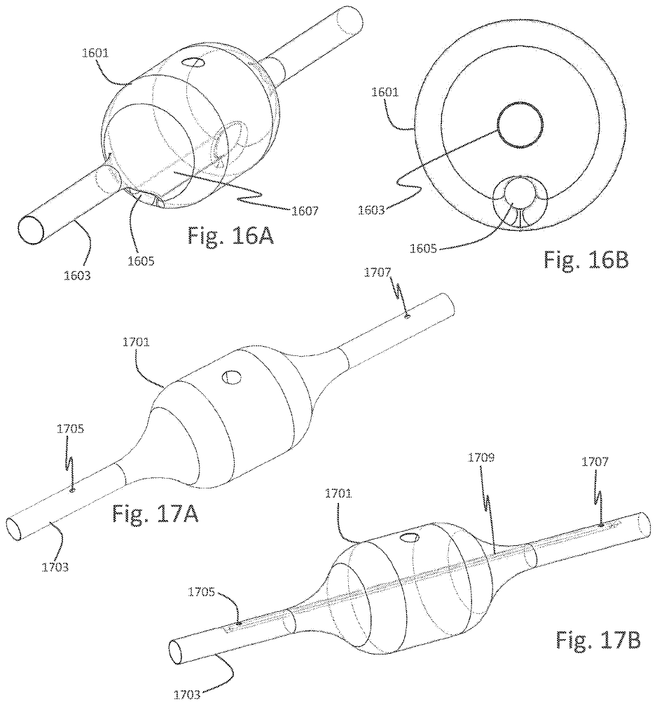

Referring to FIGS. 15A and 15B, a balloon 1501 is depicted attached to catheter shaft 1503. The balloon 1501 has an indentation or notch 1505 extending generally coaxially to catheter shaft 1503. When the balloon 1501 is inflated, blood may continue to flow in the primary vessel through the passageway 1505 formed in the balloon 1501 even while balloon 1501 occludes a branching vessel. The volume of blood passing through the balloon 1501 may be determined by a size of the lumen 1505. FIGS. 15C and 15D depict a balloon 1501 with an enlarged passageway 1505 for increased blood flow. Referring to FIGS. 16A and 16B, a balloon 1601 attached to catheter shaft 1603 is shown with an internal lumen 1607, allowing blood to flow through openings 1605 in the balloon 1601. Referring to FIGS. 17A and 17B, a balloon 1701 is attached to the outer surface of catheter shaft 1703. Catheter shaft has a first opening 1705 on one side of the balloon 1701 and a second opening 1707 on an opposite side of the balloon 1701. Catheter shaft 1703 has an interior lumen 1709 connecting the two openings 1705, 1707. Accordingly, blood may flow freely between distal and proximal ends of balloon 1701 through the catheter lumen 1709.

Radiopaque markers which may be classic marker bands or may have other shapes (symmetric or asymmetric). Additional sensors used for pressure/temperature/flow/other measurement, lesion assessment (such as Optical Coherence Tomography, Optical Coherence Reflectometry, ultrasound etc.), 3D mapping or other purposes. Such sensors may be placed distally at or near the distal end of the catheter or proximally in the handle or proximal fittings. Connectivity may be based on electrical wire, optical fibers or other.

### Procedures

In operation, step by step use of the any of the various embodiments of a catheter, e.g., catheter 100, 1300, 1400, described above may be outlined as follows. The catheter with the deflated balloons, e.g. balloon 105, is inserted inside the main vasculature 101 in the proximity of the later vein, optionally with help of an introducer/sheath, or over a guidewire. Through the use of marker bands and/or contrast fluid or other, the orientation and positioning of the coaxial double balloon 105 relative to the ostium 109 of the targeted lateral vasculature branch 103 may be controlled. In various embodiments, magnetic sensors/transducers may also be embedded, for 3D mapping purposes, for all 6 degrees of freedom (position, rotation), for use with magnetic-based systems such as, but not limited to, Biosense Webster Carto. Similarly, wired electrode rings may be included, for impedance-based mapping systems, such as, but not limited to Abbott Ensite.

The balloon catheter e.g., catheter 100, 1300, 1400, is placed next to the lateral vein 103, with the lateral port(s) 209 of the outer balloon 203 facing the ostium 109 of the vasculature 101, 103, in this aim, the inner balloon 201 may be inflated, partially or fully. The inner balloon 201 may be inflated to seal the main vasculature 101. Being compliant, or semi-compliant, and/or adequately dimensioned the outer balloon 203 stretches on the expanded shape of the inner balloon 201, complying to the inflated shape of the inner balloon. In line with the adequate orientation the device, and with the ports 209 of the outer balloon 203 overlap with the ostium 109 of the lateral branch 103. The above step may be repeated until the ports 209 of the outer balloon 203 overlap with the ostium 109 of the lateral branch 103.

A desired fluid for the medical procedure being performed (e.g., contrast fluid, saline solution, alcohol etc.), may be injected 905 through the port 205 which is connected to the empty space 301 between the two balloon layers 201, 203. As pressure builds up, the outer balloon 203 radially complies, allowing flow towards the exit port(s) 209, where the pressure equals the venous pressure. As the ports 209 are located inside the ostium 109 of the lateral vein 103, as soon as the fluid builds enough pressure to create a path 905 from the injection port 205 to the exit part 209, the fluid is injected 905 inside the lateral branch 103. After the end of the medical procedure inside the lateral branch (chemical ablation, ramification analysis through contrast fluid etc.), the inner balloon 201 is deflated and the double balloon assembly 105 is withdrawn from the vasculature 101.

The above-described methods, devices and systems for delivering infusion fluids at the ostium of a side vessel, in a manner that is superior when compared to the use presently known devices and methods.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A catheter comprising:
a shaft having an inflation lumen and an infusion lumen;
an inflation balloon having a proximal end and a distal end, each of the proximal end and the distal end secured to an outer surface of the shaft; and
an infusion balloon having a proximal end and a distal end, each of the proximal end and distal end of the infusion balloon sealed to the outer surface of the shaft, the proximal end of the infusion balloon being secured proximally of the inflation balloon and the distal end of the infusion balloon being secured distally of the inflation balloon,
wherein the shaft comprises an inflation port in fluid communication with the inflation lumen and an infusion port between the distal end of the inflation balloon and the distal end of the infusion balloon, and/or an infusion port between the proximal end of the inflation balloon and the proximal end of the infusion balloon.

2. The catheter of claim 1, wherein the inflation balloon is configured to occlude a vessel when inflated.

3. The catheter of claim 2, wherein the vessel is a coronary sinus.

4. The catheter of claim 1, wherein the infusion balloon comprises a balloon port.

5. The catheter of claim 4, wherein the balloon port is positioned to infuse a side vessel ostium.

6. The catheter of claim 5, wherein the side vessel is an oblique vein of Marshall.

7.-8. (canceled)

9. The catheter of claim 4, wherein the balloon port comprises a plurality of openings.

10. The catheter of claim 4, wherein the balloon port is defined by a reinforced radiopaque edge.

11.-12. (canceled)

13. The catheter of claim 4, wherein an infusion fluid flows from the infusion port to the balloon port through an interstitial space between the inflation balloon and the infusion balloon.

14. The catheter of claim 4, wherein an infusion fluid flows from the infusion port to the balloon port through a dedicated lumen secured to the balloon port.

15. The catheter of claim 1, wherein a plurality of bumps maintain a non-zero thickness between the infusion balloon and the inflation balloon.

16. The catheter of claim 1, further comprising an imaging device located within an interior chamber of the inflation balloon, the imaging device positioned to capture an image through the infusion balloon.

17. (canceled)

18. The catheter of claim 1, wherein a balloon lumen extends coaxially to the catheter shaft from a first opening proximal to the balloon and a second opening distal to the balloon.

19. The catheter of claim 1, wherein the inflation balloon comprises a hardened side configured to define a gap that allows the continuous circulation in the main vasculature when the inflation balloon is inflated.

20. (canceled)

21. The catheter of claim 1 suitable for positioning the catheter in the vasculature of a patient, wherein the inflation balloon is configured to be inflated to occlude a vessel and a treatment fluid can be infused into a side vessel ostium through a port in the infusion balloon.

22. A catheter comprising:
a catheter shaft comprising an inflation lumen and an infusion lumen;
a dual layer balloon mounted to an exterior surface of the catheter shaft, the dual layer balloon having an infusion port; and
an imaging device located within an interior chamber of the dual layer balloon, the imaging device positioned to capture an image through the infusion port.

23. (canceled)

24. The catheter of claim 22, wherein a light source is attached adjacent to the imaging device.

25.-31. (canceled)

32. A catheter comprising:
a catheter shaft comprising an inflation lumen and an infusion lumen, wherein a guidewire extends through the catheter shaft;
an anchor balloon;
a coaxial double balloon having an infusion port in fluid communication with the infusion lumen, the coaxial double balloon configured to translate axially in relation to the anchor balloon; and
a camera within an interior chamber of the coaxial double balloon, the camera positioned to capture an image through coaxial double balloon.

33. The catheter of claim 32, wherein the anchor balloon is distal of the coaxial double balloon.

34.-39. (canceled)

40. A method of performing fluid infusion into an ostium of a primary vein and a lateral vein, the method comprising:
guiding a catheter having a catheter shaft comprising an inflation lumen and an infusion lumen into the vasculature, wherein a dual layer balloon is mounted to an exterior surface of the catheter shaft, the dual layer balloon having an infusion port;
positioning the infusion port of dual layer balloon at the ostium;
inflating an interior balloon of the dual layer balloon such that the dual layer balloon occludes the primary vein; and
injecting an infusion fluid through the infusion port and into the ostium.
